(19) United States
(12) Patent Application Publication
(10) Pub. No.: US 2016/0100747 A1
(43) Pub. Date: Apr. 14, 2016

(54) RIGID HEAD FOR A BODY PASSAGE DEVICE

(71) Applicants: David NITSAN, Tel Aviv (IL); Nadav BEN-DA'AT, Karkur (IL); Shay DUBI, Tel Aviv (IL); JET PREP LTD., Yoqneam (IL)

(72) Inventors: David Nitsan, Tel-Aviv (IL); Nadav Ben-Da'at, Karkur (IL); Shay Dubi, Tel-Aviv (IL)

(73) Assignee: JET PREP LTD, Yoqneam (IL)

(21) Appl. No.: 14/889,770

(22) PCT Filed: May 7, 2014
(86) PCT No.: PCT/IB14/01881

§ 371 Date: Nov. 6, 2015

Related U.S. Application Data
(60) Provisional application No. 61/820,178, filed on May 7, 2013.

(51) Int. Cl. A61B 1/015; A61B 1/31; A61B 1/273; A61B 1/00; A61B 1/12

(52) U.S. Cl. CPC A61B 1/015; A61B 1/00131; A61B 1/126; A61B 1/2733; A61B 1/31

(57) ABSTRACT

A rigid head for use in a working channel of an endoscope is provided, the rigid head comprising: a body having a proximal end, a distal end and a longitudinal axis, the body having at least one channel disposed along the longitudinal axis configured to guide fluid in or out of the working channel, wherein the rigid head is configured for disposal within a working channel of an endoscope, the at least one channel configured to guide fluid in and out of the working channel of the endoscope. Body passage devices and methods are provided that allow the guidewire and/or rigid head to be extended in different length endoscope working channels.

This application claims the benefit of the filing date of U.S. Provisional Application Ser. No. 61/820,178 filed May 7, 2013. This application is incorporated herein by reference into the present disclosure.

### TECHNICAL FIELD

The present disclosure generally relates to a device for irrigating, aspirating, or cleaning patient tissue and/or the working channel or conduit of an endoscope.

### BACKGROUND

Endoscopes play a critical role in medical diagnosis and treatment. Often, endoscopes can be used to illuminate, examine and document difficult-to-access areas and other body regions to facilitate diagnosis and treatment of hidden diseases. Endoscopes can also assist in enhancing the planning and preparation of invasive operations.

Many endoscopes contain a working channel or conduit that allows the clinician to insert elongated medical devices, such as body passage cleaning devices, through the working channel and into a body passage (e.g., colon, esophagus, etc.). These elongated medical devices can be used to irrigate, clean, biopsy, repair or remove patient tissue. Some endoscopes have working channels that allow insertion of elongated medical devices for irrigation, aspiration and/or cleaning of patient tissue or for the removal of debris from the working channel of the endoscope.

Some elongated medical devices have spray heads on their distal ends that make irrigation and/or cleaning of the working channel or patient tissue easier. These spray heads are often disposed on a guidewire that allows the clinician to move the spray head in and out of the working channel of the endoscope as well as beyond the endoscope to the patient tissue or body cavity (e.g., colon, esophagus, etc.). These spray heads are deformable and expand and contract within the working channel of an endoscope.

Unfortunately, there can also be unwanted lateral or vertical movement of the guidewire and/or the spray head relative to the working channel and any tube surrounding the guidewire. This unwanted movement can reduce the precision in irrigating or cleaning of the working channel or patient tissue. There can also be less precision when positioning the elongated medical device relative to other medical devices that can also be disposed in the working channel of the endoscope.

Therefore, there is a need for a spray head that is rigid that can be disposed in and out of a working channel of an endoscope for irrigating and/or cleaning the working channel of an endoscope or patient tissue. Body passage devices and methods that have a rigid spray head disposed on a guidewire that can extend in and out of a working channel of an endoscope would also be beneficial.

### SUMMARY

New spray heads that are rigid are provided and can be disposed in and out of a working channel of an endoscope for irrigating and/or cleaning the working channel of an endoscope or patient tissue. Body passage devices and methods that have a rigid spray head disposed on a guidewire that can extend in and out of a working channel of an endoscope are also provided.

The spray heads that are rigid can be inserted into a working channel of an endoscope to clean, aspirate or irrigate the working channel or a certain patient tissue (e.g., colon, esophagus, etc.). This will allow removal of debris surrounding the endoscope and viewing the patient tissue will be clearer to better help diagnosis and treatment of the patient.

In one embodiment, there is a rigid head for use in a working channel of an endoscope, the rigid head comprising: a body having a proximal end, a distal end and a longitudinal axis, the body having at least one channel disposed along the longitudinal axis configured to guide fluid in or out of the working channel, wherein the rigid head is configured for disposal within a working channel of an endoscope, the at least one channel configured to guide fluid in and out of the working channel of the endoscope.

In one embodiment, there is a body passage device, comprising a housing configured to receive a guidewire, the housing having a longitudinal axis; a first guidewire adjuster contacting the housing and having a proximal end and a distal end, the first guidewire adjuster configured to extend the guidewire a first length along the longitudinal axis of the housing; and a second guidewire adjuster configured to contact the proximal end and/or the distal end of the first guidewire adjuster and/or guidewire and to extend the guidewire a second length along the longitudinal axis of the housing. The guidewire has a rigid spray head attached thereto.

In one embodiment, in accordance with the principles of the present disclosure, a system for cleansing tissue is disclosed. The system comprises a rigid head unit comprising a body having a proximal face, a distal face and a channel extending between the proximal face and the distal face. The head defines a longitudinal axis between the proximal face and the distal face. The head is attached to a distal end of a guidewire extending from an irrigation tool. The system further includes a working channel extending between the rigid head and the irrigation tool, wherein the proximal face is connectable to the working channel of the surgical tool for transmission of matter.

In one embodiment, in accordance with the principles of the present disclosure, a kit for cleansing tissue is disclosed. The kit comprises an irrigation tool, a rigid head unit, and a working channel. The irrigation tool is configured to receive a guidewire and comprises a housing having a longitudinal axis. A first guidewire adjuster contacts the housing and has a proximal end and a distal end. The first guidewire adjuster is configured to extend the guidewire a first length along the longitudinal axis of the housing. A second guidewire adjuster is configured to contact the proximal end and/or the distal end of the first guidewire adjuster and/or guidewire and to extend the guidewire a second length along the longitudinal axis of the housing. The rigid head comprises a body having a proximal end and a distal end and a longitudinal axis. The body has at least one channel disposed along the longitudinal axis. The rigid head is configured to be disposed within a working channel of an endoscope. The at least one channel is configured to guide fluid in or out of the working channel. The proximal face of the body is connectable to the surgical tool for transmission of matter. The working channel extends between the rigid head and the irrigation tool. The working channel comprises an element on an inner surface to provide a tight fit between an outer surface of the rigid head unit and the element.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

In part, other aspects, features, benefits and advantages of the embodiments will be apparent with regard to the following description, appended claims and accompanying drawings where:

FIG. 1 is a side, breakaway view of an embodiment of a body passage device in accordance with the principles of the present disclosure;

FIG. 2 is a perspective view of components of an embodiment of the body passage device shown in FIG. 1;

FIG. 3 is a side cross-sectional view of components of an embodiment of the body passage device shown in FIG. 1;

FIG. 4 is a perspective view of components of an embodiment of the body passage device shown in FIG. 1;

FIG. 4A is a perspective view of components of an embodiment of the body passage device shown in FIG. 1;

FIG. 5 is a side cross-sectional view of components of an embodiment of the body passage device shown in FIG. 1;

FIG. 6 is a perspective view of components of an embodiment of the body passage device shown in FIG. 1;

FIG. 7 is a perspective view of components of embodiments of the body passage device shown in FIG. 1;

FIG. 8 is a cross sectional plan view of components of the body passage device shown in FIG. 1;

FIG. 9 is a cross sectional plan view of components of the body passage device shown in FIG. 1;

FIG. 10 is a cross sectional view of components for use with the body passage device shown in FIG. 1;

FIG. 11 is a cross sectional view of components for use with the body passage device shown in FIG. 1;

FIG. 12 is a cross sectional view of components for use with the body passage device shown in FIG. 1;

FIG. 13 is a cross sectional view of components for use with the body passage device shown in FIG. 1;

FIG. 14 is a cross sectional view of components for use with the body passage device shown in FIG. 1;

FIG. 15 is a cross sectional view of components for use with the body passage device shown in FIG. 1;

FIG. 16 is a perspective breakaway view of components of the body passage device shown in FIG. 1;

FIG. 17 is a side cross-sectional view of components of the body passage device shown in FIG. 1;

FIG. 18 is a side, breakaway view of an embodiment of a device in accordance with the principles of the present disclosure; and

FIG. 19 is a side, breakaway view of an embodiment of a device in accordance with the principles of the present disclosure.

Like reference numerals indicate similar parts throughout the figures. It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

New spray heads that are rigid are provided and can be disposed in and out of a working channel of an endoscope for irrigating and/or cleaning the working channel of an endoscope or patient tissue. Body passage devices and methods that have a rigid spray head disposed on a guidewire that can extend in and out of a working channel of an endoscope are also provided.

The spray heads that are rigid can be inserted into a working channel of an endoscope to clean, aspirate or irrigate the working channel or a certain patient tissue (e.g., colon, esophagus, etc.). This will allow removal of debris surrounding the endoscope and viewing the patient tissue will be clearer to better help diagnosis and treatment of the patient.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

The following discussion includes a description of a body passage cleaning device and related methods of employing the body passage cleaning device in accordance with the principles of the present disclosure. Alternate embodiments are also disclosed. Reference is made in detail to the exemplary embodiments of the present disclosure, which are illustrated in the accompanying figures.

This disclosure describes an improvement over these prior art technologies including those body passage devices described in PCT/IL2009/000346 filed on Mar. 26, 2009, U.S. application Ser. No. 12/923,796 filed Oct. 7, 2010 and PCT/IL2011/000086 filed Jan. 11, 2011. These entire disclosures are herein incorporated by reference into the present disclosure.

It should be noted that while this disclosure refers mainly to the use of the present disclosure within the context of colonic cleansing and colonoscopic procedures, the devices and systems disclosed herein may equally be used in connection with other endoscopic devices, in other body passages, such as in different regions of the gastro-intestinal tract or in the respiratory tract with a bronchoscope.

The present disclosure also provides a colonic cleansing system comprising some or all of the features disclosed in the aforementioned co-owned published international patent applications (PCT/IL2009/000346 filed on Mar. 26, 2009, U.S. application Ser. No. 12/923,796 filed Oct. 7, 2010 and PCT/IL2011/000086 filed Jan. 11, 2011, all of which are incorporated herein by reference), but in which the prior art elastically-deformable spray head unit is replaced by the presently-disclosed rigid head unit. In its most general form, the colonic cleansing system of the present disclosure comprises a rigid spray head mounted on the distal end of an elongate actuating element (such as a guidewire), wherein said actuating element may optionally be partially or completely surrounded by one or more concentrically-arranged conduits. The system will also comprise a proximal control handle for use in controlling inter alia the proximal-to-distal flow of irrigation fluid (supplied by an external pump and fluid reservoir) and aspiration of said fluid together with dislodged debris in the reverse direction. The proximal control handle is also used for controlling the position of the rigid head unit within, and beyond the distal end of, the colonoscopic working channel. A complete description of some suitable types of proximal handles is contained within the aforementioned co-owned published patent applications.

The rigid head unit and colonic cleansing system of the present disclosure may be used to clean the colon during colonoscopic procedures by means of performing, and/or enabling the performance of, the following three functions when inserted into the colon (or other intestinal region) of a subject: (a) irrigation of the lumen of the body passage in which the endoscope is placed, distal to the distal end of said endoscope; (b) aspiration of the irrigation fluid (and associated debris) from the irrigated region, in a proximal direction, through the working channel (or other internal space of the endoscope; and (c) clearance of debris from the working channel, in a proximal direction, in order to prevent build-up of solid and semi-solid matter which would be liable to cause blockage of said working channel.

While the first two of these functions require the presence of longitudinally-disposed apertures or channels that connect the colonic environment distal to the end of the colonoscope with the lumen of the colonoscopic working channel, the third function—channel clearance—requires that the distal end of the working channel become sealed. In this way, negative pressure applied at the proximal end of the working channel will cause the evacuation of the contents of said channel, without causing the aspiration of additional fluid and solid material from the colon itself.

In some embodiments of the present disclosure, the rigid head unit is intended for use within a standard, unmodified, rigid-wall working channel. In other embodiments, the distal portion of the colonoscopic working channel has been modified such that it comprises an elastically-deformable element or other means for improving its ability to form a fluid-impermeable seal between the distal portion of said working channel and the rigid distal head unit inserted therein. Details of these two main variants of the disclosure will be provided hereinbelow.

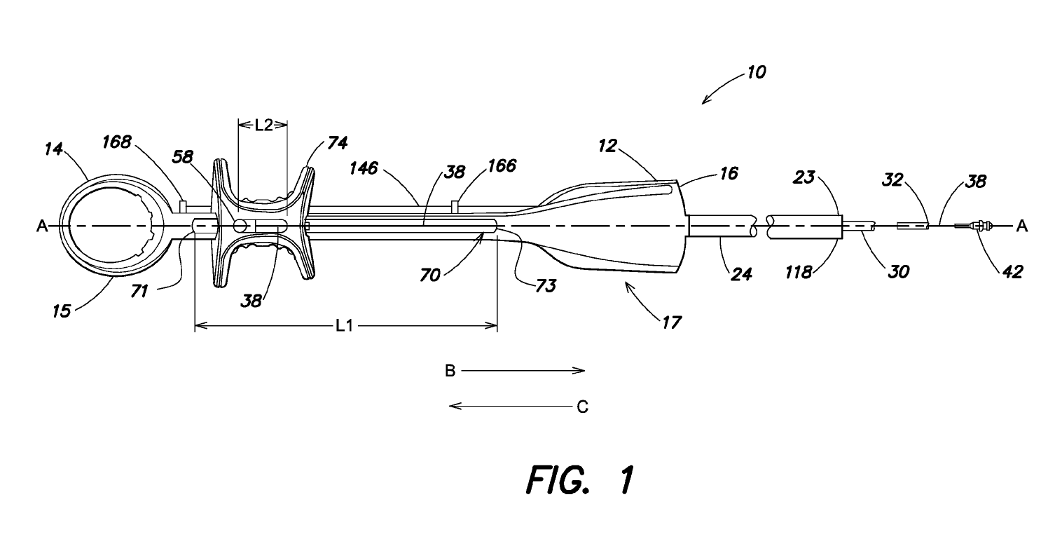

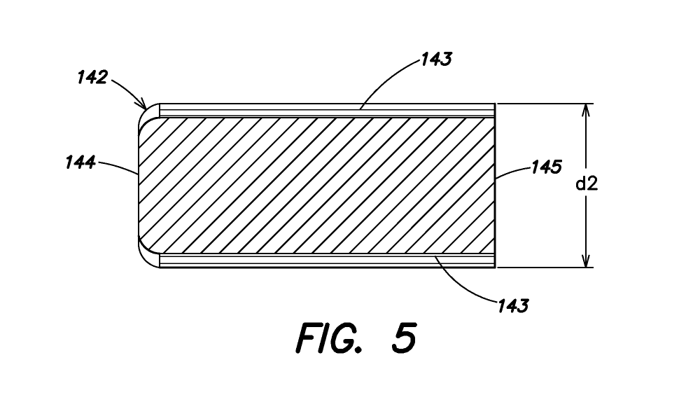

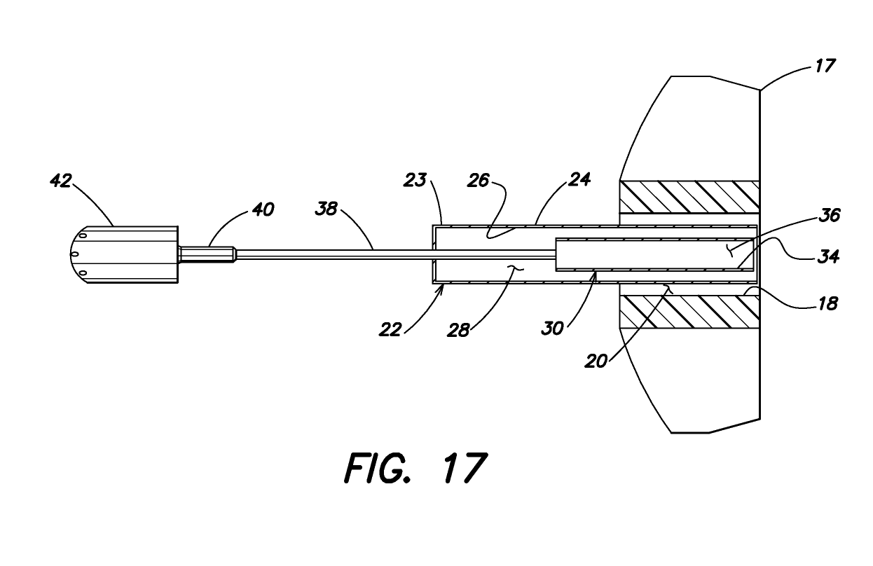

Turning to FIGS. 1-19, there are illustrated components of a body passage cleaning device 10. The components of device 10 can be fabricated from biologically acceptable materials suitable for medical applications, including metals, synthetic polymers, ceramics, or composites. For example, the components of device 10, individually or collectively, can be fabricated from materials such as machined metal and/or machined or injection molded plastic, stainless steel alloys, commercially pure titanium, titanium alloys, Grade 5 titanium, super-elastic titanium alloys, cobalt-chrome alloys, stainless steel alloys, super-elastic metallic alloys (e.g., Nitinol, super elasto-plastic metals, such as GUM METAL® manufactured by Toyota Material Incorporated of Japan), ceramics, thermoplastics such as polyaryletherketone (PAEK) including polyetheretherketone (PEEK), polyetherketoneketone (PEKK) and polyetherketone (PEK), carbon-PEEK composites, PEEK-BaS04 polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyetherimide, polyethylene, epoxy, partially resorbable materials, totally resorbable materials, polyglycolide, polytyrosine carbonate, polycaroplaetohe and their combinations, bioactive glasses, porous metals, or any combination thereof.

Various components of device 10 may have material composites, including the above materials, to achieve various desired characteristics such as strength, rigidity, elasticity, compliance, biomechanical performance, durability and radiolucency or imaging preference. The components of device 10, individually or collectively, may also be fabricated from a heterogeneous material such as a combination of two or more of the above-described materials. The components of device 10 may be monolithically formed, integrally connected or include fastening elements and/or instruments, as described herein.

Device 10 is configured to be introduced through the working channel of a medical device, such as, for example, an endoscope to cleanse a body cavity, such as, for example, the colon of a patient. Device 10 includes a housing, such as, for example, a handle 12 extending along a longitudinal axis A between a distal end 14 defining a thumb ring 15, and a proximal end 16 opposite end 14 that defines a manifold 17. Handle 12 includes an inner surface 18 defining an elongated cavity 20. Handle 12 may be manufactured from ABS, polycarbonate, Delrin and other plastic resins depending on the compatibility with the sterilization method to be used (e.g., autoclaving, Gamma radiation or ETO, etc.) preferably, by means of casting in mass production. The length of handle 12 may generally be about 80-120 mm.

In some embodiments, handle 12 includes one or more passages for connecting a fluid-supply channel to one, two or more fluid outlet channels. In some embodiments, handle 12 includes means for switching between the fluid outlet channels to which said fluid-supply channel is connected. In some embodiments, handle 12 includes one fluid outlet channel in fluid communication with a working channel, such, as, for example, a passageway 32 of an inner tube 30, and a second fluid outlet channel in fluid communication with the space surrounding the external surface of tube 30. When inserted into a working channel of an endoscope, this latter space will be bound externally by the walls of the working channel. In some embodiments, handle 12 may be used to operate endoscopic instruments, such as a colonic cleansing device of the present disclosure. In such embodiments, handle 12 incorporates a mechanism for directing flow to different lumens and conduits. It is to be emphasized that in addition to its use in conjunction with the colonic cleaning device incorporating tube 30, handle 12 may also be used together with variants of this device (e.g. not comprising an outer tube) and indeed with other endoscopic instruments. In some embodiments of handle 12, the aforementioned switching means comprises a multi-way fluid valve. In some embodiments, the coupling means comprises a mechanical actuator (for example in the form of an elongated strip or bar) which is connected to both a slider mechanism and to the aforementioned switching means. In some embodiments, tube 30 may be made from ETFE, PTFE or other plastic resins which are compatible with the applicable sterilization method with low friction coefficient and sufficient rigidity to support the device without collapsing. In some embodiments, tube 30 may be made of silicon or rubber resin, preferably from PTFE for low friction between the guidewire and tube as well as between tube 30 and the working channel, having a length of about 100 cm-210 cm, depending on the endoscope length used, as well as the external extension tubing length, and having an inner diameter generally in the range of 1 mm or smaller to reduce hysteresis effects (around AWG16), and a wall thickness of about 0.25 mm to 0.4 mm.

A tube 22 is positioned in cavity 20 such that an outer surface 24 of tube 22 engages surface 18. Tube 22 enables device 10 to function either within a working channel of an endoscope (or any other instrumental lumen) or within a naturally-occurring channel or passageway (such as the colonic lumen). Tube 22 provides device 10 with the following additional advantages: a) a stand-alone configuration which permits the use of the device in any lumen (natural or instrumental) including endoscopic working channels; b) device 10 may extend beyond the distal end of the working channel and/or other natural or instrumental lumen; c) a camera may be included on tube 22 to enable viewing of further areas while performing the cleansing; d) aspiration and irrigation will be done through the gap between tube 22 and tube 30. In some embodiments, tube 22 may be constructed such that it has a flexible, semi rigid or rigid configuration. In some embodiments, tube 22 may be made from ETFE, PTFE, and Nylon etc., preferably from PTFE, has a length of about 50-70 cm, depending on the endoscope length used as well as the external extension tubing length, an inner diameter generally similar to the working channel diameter in the range of 2 mm-4 mm or smaller to reduce hysteresis effects (around AWG8), and wall thickness of about 0.5-1 mm.

Tube 22 is fixed relative to handle 12. An end 23 of tube 22 extends beyond manifold 17 such that end 23 is positioned outside of cavity 20. Tube 22 is larger than the guidewire and spans around it in 118. In some embodiments, tube 22 can be variously connected with handle 12, such as, for example, monolithic, integral connection, frictional engagement, threaded engagement, mutual grooves, screws, adhesive, or raised element. In some embodiments, tube 22 comprises a flexible material to allow tube 22 to bend during insertion into and/or translation through a lumen of a patient's body, such as, for example, the patient's colon. In some embodiments, tube 22 comprises a transparent or translucent material to facilitate viewing of components disposed within tube 22.

In some embodiments, tube 22 is constructed from a non-compliant material such as nylon, Pebax (or a blend thereof), polyurethane and polyethylene terephthalate (PET). In such a case, tube 22 has a random flat shape when in an unexpanded configuration, which becomes circular in cross section when expanded with irrigation fluid. In some embodiments, tube 22 is made from a compliant material such as silicone or a thermoplastic elastomer (TPE), wherein tube 22 is able to expand in the same manner as a compliant balloon. In some embodiments, tube 22 may be made from ETFE, PTFE, and Nylon etc., preferably from PTFE, having a length of about 50-70 cm, depending on the endoscope length used as well as the external extension tubing length, an inner diameter generally similar to the working channel diameter in the range of 2 mm-4 mm or smaller to reduce hysteresis effects (around AWG8), and wall thickness of about 0.5-1 mm.

In some embodiments, tube 22 includes a lumen extending parallel to passageway 28 running along the length of the tube 22. This additional lumen 36 may be used for a number of purposes including: injection of therapeutic agents, injection of iodine (for chromo-endoscopy), application of very cold water in order to arrest bleeding, and delivery of tumor-specific bio-markers. In addition, the extra lumen may be used to introduce air for insufflation of air mixture with the irrigation fluid. In some embodiments, the additional lumen may have a very small diameter (e.g. 0.2-1 mm) and may be contained within a collapsible tube. The additional tube, in some embodiments, may reduce lateral motion of the guidewire and the rigid head.

In some embodiments, tube 22 may allow fluid to be conducted a long its interior 26. Thus fluid will flow out of the tube 22 and contact rigid head 42 and be guided through channels in rigid head 42. In some embodiments, tube 22 does not change its diameter under the liquid pressure (e.g., it is non-collapsible). Such a configuration may be advantageous when it is required mainly to irrigate a specific location without necessarily allocating more space to the working channel. This configuration may also be advantageously employed in situations wherein the space between the working channel and the lumen of the irrigation water is sufficiently large to allow aspiration and/or insertion of additional tooling through the working channel.

This particular embodiment, while employing a conventional catheter tube is characterized by the following notable features: a) miniaturization of a rigid head 42 to 2-3 mm overall diameter to enable insertion through small lumens such as the working channel of an endoscope (e.g., colonoscope; etc.) b) the irrigation fluid is directed through channels or nozzles in rigid head 42; c) it is required to design specific nozzles to optimize the pressure flow and avoid turbulences wherever possible; and d) enables maximum liquid momentum with minimal liquid volume. For example, if the irrigation liquid is transformed to a spray where the liquid drops are very small no cleansing effects can be achieved. Alternatively, if the nozzle is too large (e.g. 0.8 mm-3.8 mm) to provide the cleansing momentum required for irrigation an impractically-large volume of irrigation fluid will be required. The irrigation nozzles are focused forward (in an inward, outward angles or straight), thus enabling the physician maximum efficiency and maximum effective force (liquid momentum) in the direction where the colonoscope camera is directed. Automatic cleansing in 320° enables irrigation where no vision is available (for example, out of the field of view or in diverticulosis).

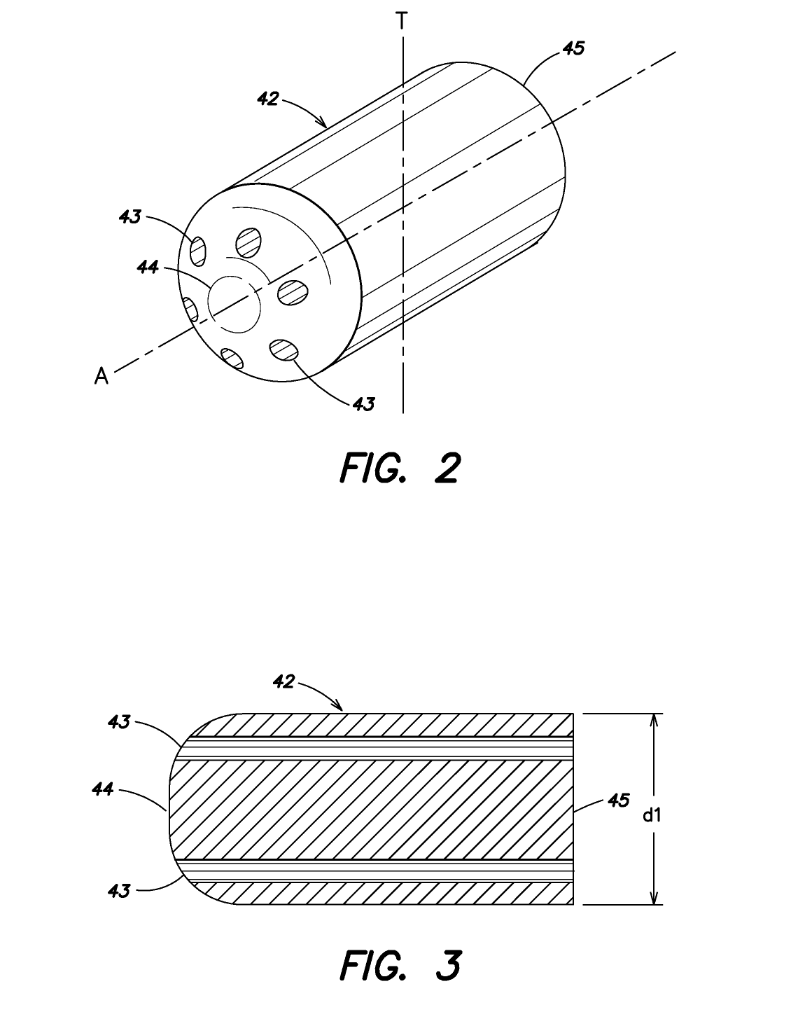

An inner surface 34 of tube 30 defines passageway 32 having a cylindrical cross sectional configuration and configured for movable disposal of an actuating element, such as, for example, a guidewire 38. In some embodiments, guidewire 38 has a length that is greater than that of tube 30, such that a portion of guidewire 38 is left unenclosed by tube 30. Guidewire 38 includes an end 40 defining the rigid head 42. In one embodiment, rigid head 42 includes a plurality of channels, apertures and/or nozzles which are capable of allowing the passage of a fluid therethrough, as discussed herein. At least an outer portion of rigid head 42 is capable of retaining its shape when compression forces are exerted thereon. The channels, apertures and/or nozzles of rigid head 42 are in always open when the rigid head 42 is subjected to said compression forces in the working channel such that the fluid is capable of passing through the channels, apertures and/or nozzles. Suitable material and configurations for the spray head 42 can be found in PCT/IL2009/000346 filed on Mar. 26, 2009, U.S. application Ser. No. 12/923,796 filed Oct. 7, 2010 and PCT/IL2011/000086 filed Jan. 11, 2011, which are each incorporated herein by reference, in their entireties. However, the spray heads mentioned in these applications are not rigid. Further embodiments of rigid spray heads of the present disclosure are described in FIGS. 2-7.

In some embodiments, guidewire 38 comprises a flexible material to allow guidewire 38 to bend during insertion into and/or translation through a lumen of a patient's body, such as, for example, the patient's colon. In some embodiments, guidewire 38 can contain one or more radiopaque materials at various points along its length (not shown). These radiopaque markers may be used to locate the device by means of real time visualization using X-ray imaging. The use of such markers is particularly important in upper GI endoscopic procedures, for example when introducing endoscopic tooling trough the papilla. In some embodiments, guidewire 38 has a diameter of about 0.25 mm to about 0.6 mm. In some embodiments, guidewire 38 has a diameter of about 0.3 mm to about 0.8 mm. In some embodiments, guidewire 38 has a diameter of about 0.4 mm to about 1.5 mm. In some embodiments, guidewire 38 is made from stainless steel 304V with an optional configuration of PTFE coating to reduce potential friction between guidewire 38 and tube 30 and its diameter may generally be in range of 0.5-0.7 mm, or about 0.6 mm.

In some embodiments, passageway 32 defines a channel configured for both passing irrigation fluid distally and aspirating fluid and solid debris proximally, wherein both of these processes may be performed in a highly effective manner as part of a procedure for cleansing internal body passages and cavities. This configuration permits effective and higher-pressure irrigation of a body cavity via an endoscopic working channel while still allowing for the use of the same working channel for other purposes, most particularly the aspiration of fluid and debris from said body cavity. This configuration also permits irrigation and cleansing of the working channel of an endoscopic instrument without the removal of the instrument from the body, such that blockages of the channel by fecal material and debris may be prevented or removed. This configuration further permits irrigation, cleansing and aspiration for additional applications such as, for example, upper and lower GI bleeding, bronchoscopy, cystoscopy, gastrostomy trauma surgery where no preparation was available and endo-surgery preparation and additionally permits the upgrading of all endoscopic devices by integrating them together with a nozzle assembly, thereby not requiring the replacement of tools (for example biopsy forceps, snares, injection needles, and so on) during the procedure. In some embodiments, to pump irrigation fluid into passageway 32, tube 30 may be connected to a positive pressure liquid pump (centrifugal, peristaltic or other) or to a manual injector. To cause collapse of tube 30, it can be connected to a manual injector or through a negative pressure vacuum pump.

In some embodiments, tube 30 is used in an endoscope working channel (or similar instrumental lumen). An attachment seal is inserted around tube 30 at the proximal entrance to passageway 32. This sealing element, which may be constructed of a flexible material such as a biocompatible rubber, plastic or metal, prevents irrigation and debris being sprayed on the operator. In addition, its presence is necessary for maintaining integrity of the negative aspiration pressure that is applied to the space between tube 22 and tube 30. The presence of tube 30 in device 10 results in liquid and debris aspiration taking place through device 10 (in the space between tubes 22, 30) rather than through the working channel itself. This arrangement therefore requires the inclusion of a Y-connector at the proximal end of the working channel, in order to direct the aspirated liquid and solid material—via a one-way valve—along a waste line to a collection container.

In some embodiments, tube 30 comprises a collapsible sheath. A proximal end of tube 30 is connected to a source of irrigation fluid (e.g. saline) and suitable pumping apparatus. When the irrigation fluid is pumped through passageway 32, the tube 30 moves from a flaccid, collapsed state to a fully expanded conformation, thereby allowing maximum transfer of the irrigation fluid to the rigid head 42, details of which are discussed hereinbelow. When the irrigation fluid ceases to flow through tube 30 (e.g. as a result of both turning off the irrigation fluid pump and connecting the proximal end of tube 30 to a negative pressure source), tube 30 loses its source of structural rigidity (the column of irrigation fluid) and returns to the flaccid, collapsed state, thereby increasing the volume of a working channel that is external to the irrigation catheter. This is highly advantageous, for at least the following three reasons: a) maximum space for aspiration of irrigation fluid and fecal debris through the working channel is provided; b) additional space for the introduction and passage of endoscopic surgical tools (without the need to remove the irrigation catheter) is created; c) insufflation of the body cavity (e.g., colon) may be performed in the presence of tube 30, when collapsed. In some embodiments, irrigation fluid is pumped through passageway 32 under positive pressure, said fluid is directed to the body cavity that is being cleansed (e.g. the colon). Conversely, when a negative pressure source is connected to the proximal end of tube 30, tube 30 collapses, thereby creating a larger free volume within passageway 32. In order to create optimal sheath collapse, a one way valve may be used.

In FIG. 1, there is also shown the device with other component parts, which can be used to extend the rigid head 42 in and out of the working channel of an endoscope. Button 58 extends through a groove in an external slider 74 that is movable along a handle to move slider and thus the guidewire and the rigid head 42 in directions shown by arrows B or C. Button 58 is movable in distance L2 for additional length for the guidewire. The guidewire is disposed in groove 70 and extends out end 73. Groove 70 has a length L1 defined by a distance between an end 71 of groove 70 and an end 73 of groove 70. The device may include a distal stop element 166 and a proximal stop element 168, stop elements 166, 168 being designed to be respectively pushed and pulled by slider 74. A distal end of rod 146 is fitted with a pair of jaws (not shown) which are used to grasp the device and ease in movement of its components.

In some embodiments, the term “distal spray head unit” or the like are sometimes used interchangeably with the terms “distal plug,” “spray head” or “rigid head.” It should further be noted that the term “distal” refers to the direction away from the operator and towards the patient's body. Consequently, the term “proximal” is taken to refer to the opposite direction. In some embodiments, the external diameter of distal head 42 is larger than the internal diameter of passageway 32. In some embodiments, the internal diameter of passageway 32 is 3.8 mm or the range of 2-4 mm.

In some embodiments, since the distal-proximal location of guidewire 38 is altered during use, while the position of tube 30 is fixed (with reference handle 12), the precise distance between the distal end of tube 30 and the more distally placed distal end of guidewire 38 will also alter, and will generally be in the range of about 1 cm to about 4 cm. In general, the total length of guidewire 38 (which is typically constructed of 0.5-0.6 mm diameter) will be in the range of about 150 cm-210 cm, depending on the endoscope length used as well as the external extension tubing length. Extension tubing is assembled between the endoscope working channel adapter and the handheld device, having a length of 50-70 cm with an internal diameter of similar to the working channel diameter (3.8 mm). In some embodiments, the distal end of guidewire 38 is attached to the distal spray head unit by means of gluing, bonding or laser welding/soldering of the metal guide wire and the distal plug. Tube 30 is constructed of PTFE tubing (for low friction) or ETFE, depending on the sterilization method of the device to be used, and has an external diameter of about 1 mm-1.6 mm and a wall thickness of about 0.25 mm. It is to be recognized, however, that these measurements are given only as a general guide and do not limit the scope of the present disclosure in any way.

In some embodiments, rigid head 42 is constructed such that it may be caused to move between two conformations. In the first conformation, wherein rigid head 42 has a size that permits its distal passage through passageway 28 prior to irrigation and aspiration, and its proximal passage following the end of those procedures. This conformation is also used for sealing the distal exit of passageway 32, such that it may assist in cleansing an endoscope working channel 80 utilizing tube 30 (the distal end of which ends a few centimeters from the distal end of guidewire 38) to supply high pressure irrigation fluid to the distal part of passageway 32. In this way, positive hydrostatic pressure forces are added to the vacuum pressure, thereby significantly increasing the efficiency with which particulate matter may be moved proximally from the distal end of passageway 32 and thus preventing and/or clearing blockages therein.

Generally, this first conformation is adopted when device 10 is contained within the confines of the passageway 32 (other narrow instrument channel). The external diameter of rigid head 42 is generally constructed to be only larger than the internal diameter of passageway 32, such that when rigid head 42 is contained within passageway 32, the outer surface of rigid head 42 tightly fits with the inner surface of passageway 32. The second conformation occurs when rigid head 42 leaves the confines of passageway 32 (at its distal end). A proximally-directed force is then applied such that rigid head 42 makes contact with the distal exit of passageway 32, effectively providing a fluid seal over said exit, such that the only fluid transfer between passageway 32 and the region of the body cavity located beyond the distal end thereof is by way of channels, apertures and/or nozzles of the rigid head 42. In some embodiments, the first and second conformations are incorporated into a single rigid head 42.

The rigid head is made from rigid or substantially rigid material. It should be noted that the term rigid material as used herein is meant to encompass rigid materials that have little or no flexibility. For example, the rigid material may be metal, carbon fiber, composites, ceramics, plastic, or the like. The rigid material, in some embodiments, will have little or no flexibility in the working channel of the endoscope so that the channels disposed on or in the rigid head will not collapse on themselves and are, therefore, in the open position to act as a guide to allow fluid (e.g., liquid or gas) to flow in and/or out of the channel. The rigid head may span across one or more dimensions of the endoscope and/or working channel (e.g., across a width, length, and/or height of the working channel). In some instances, the rigid head may represent a three-dimensional object (e.g., a molded or machined component), or the like.

Rigid head 42 of the present disclosure, in one embodiment, is constructed as an entirely rigid unit, having a length in the range of 2-20 mm, comprising one or more small-diameter longitudinally-disposed irrigation channels, such as, for example, channels 43 and having a maximum diameter, such as, for example, diameter d1, very similar to, and just smaller than, the inner diameter of a working channel 80 of an endoscope through which it is passed. In this way, when placed within the distal portion of passageway 32, the close tolerance with respect to diameter d1 of rigid head 42 will permit a pressure head of fluid to build up proximal to rigid head 42 and thereby allow a fluid spray to pass through channels 43 into the colonic lumen, thereby irrigating that region of the colon. Conversely, application of suction pressure to the proximal end of the working channel and further distal movement of the rigid head 42 such that it leaves the confines of working channel 80 and passes within the adjacent region of the colonic lumen, will cause efficient, large-volume aspiration of fluid, solid and semi-solid debris in a proximal direction through the working channel.

In one embodiment, diameter d1 of rigid head 42 unit is very close to the diameter of working channel 80 of the endoscope through which rigid head 42 is passed. Thus, the dimensions selected should be such that they permit low friction between rigid head 42 and working channel 80 during insertion and removal of rigid head 42 through the endoscope and in/out of the endoscope distal portion. In some embodiments, in order to enable adequate irrigation, rigid head 42 should seal at least 60%, at least 65%, at least 70%, at least 75%, at least 80%, at least 90%, or at least 95% of the endoscope working channel outlet surface and therefore must be close in diameter to working channel 80. However, in some embodiments, in order to permit sufficiently free movement of rigid head 42 within working channel 80, the largest diameter d1 of rigid head 42 is at least 0.1 mm less than an inner diameter of working channel 80. Thus, diameter d1 of a rigid head 42 is dependent on the diameter of working channel 80 in conjunction with which rigid head 42 will be used. Since endoscope working channels normally have a diameter in the range of 2 mm to 6 mm, the rigid head 42 of the present disclosure generally comprise a diameter in the range of about 1.9 mm to about 5.9 mm.

Rigid head 42 extends along longitudinal axis A between a distal face 44 and a proximal face 45. In one embodiment, distal face 44 comprises a bullet shape, and proximal face 45 comprises a flat shape parallel to transverse axis T. In one embodiment, both distal face 44 and proximal face 45 comprise bullet shapes to facilitate movement into and out of working channel 80 and implantation into tissue. In one embodiment, both distal face 44 and proximal face 45 comprise flattened configurations parallel to transverse axis T. In one embodiment, as shown in FIG. 2, the irrigation channels 43 are formed such that they perforate proximal face 45 of rigid head 42. In one embodiment, channels 43 pass through the entire length of rigid head 42, perforating distal face 44. In the example shown in FIG. 2, rigid head 42 contains six irrigation channels arranged in a circular pattern. However, this embodiment is meant to be illustrative only, and rigid head 42 may contain fewer or greater numbers of such channels in varying arrangements. In one embodiment, channels 43 comprise a circular cross-section. In some embodiments, channels 43 can be variously configured, such as, for example, oval, oblong, triangular, square, polygonal, irregular, uniform, non-uniform and/or tapered.

Rigid head 42, in the embodiment shown, comprises a generally cylindrical shape and includes diameter d1 along its length. In various embodiments, diameter d1 of rigid head 42 may increase or decrease along its length, as may be required by a particular application. In one embodiment, rigid head 42 has a frictional surface configuration for engagement with working channel 80 to enhance fitting. In some embodiments, the surface of rigid head 42 may include alternate surface configurations, such as, for example, smooth, rough, arcuate, undulating, mesh, porous, semi-porous, dimpled and/or textured according to the requirements of a particular application.

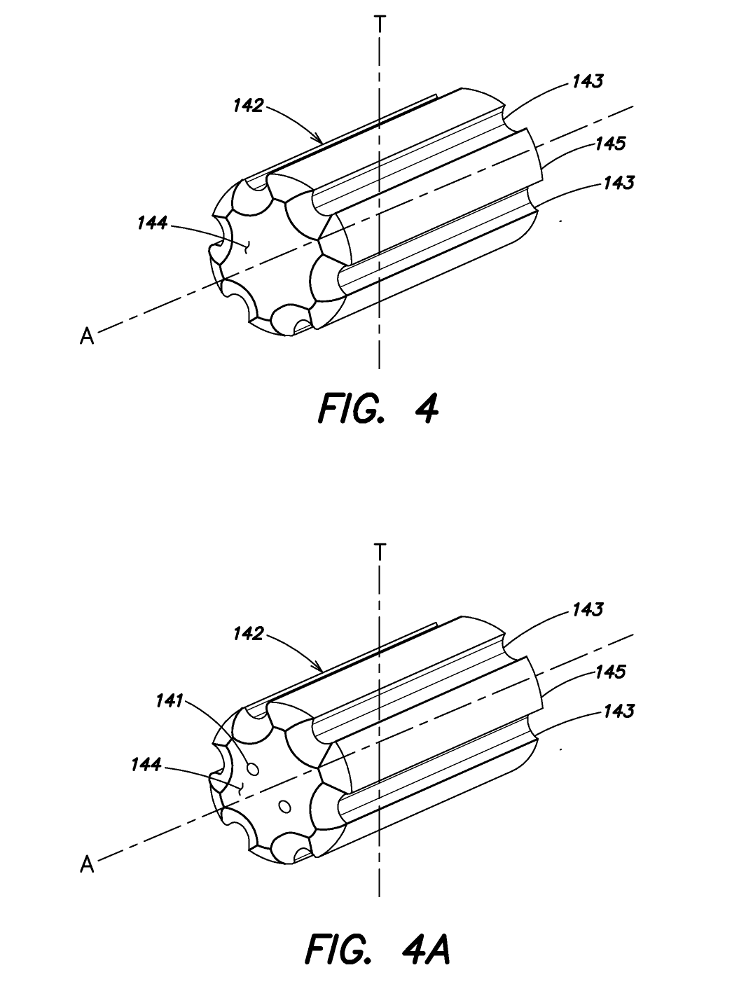

Similar to rigid head 42, spray head 142 extends along longitudinal axis A between a distal face 144 and a proximal face 145. In one embodiment, distal face 144 comprises a bullet shape, and proximal face 145 comprises a flat shape parallel to transverse axis T. In one embodiment, both distal face 144 and proximal face 145 comprise bullet shapes to facilitate movement into and out of working channel 80 and implantation into tissue. In one embodiment, both distal face 144 and proximal face 145 comprise flattened configurations parallel to transverse axis T. In one embodiment, as shown in FIG. 4, channels 143 are formed as grooves around the circumference of spray head 142. As a consequence, only a portion of the wall of each channel 143 is formed by spray head 142 itself, with the remainder of each channel 143 wall being formed by the wall of the working channel when spray head 142 is inserted into working channel 80 of an endoscope.

As shown in FIG. 4, spray head 142 contains six channels. However, this embodiment is meant to be illustrative only, and spray head 142 may contain fewer or greater numbers of such channels in varying arrangements. In one embodiment, channels 143 comprise a circular cross-section. In some embodiments, channels 143 can be variously configured, such as, for example, oval, oblong, triangular, square, polygonal, irregular, uniform, non-uniform and/or tapered.

Spray head 142 comprises a generally cylindrical shape and includes a diameter d2 along its length. In various embodiments, diameter d2 of spray head 142 may increase or decrease along its length, as may be required by a particular application. In one embodiment, spray head 142 has a frictional surface configuration for engagement with working channel 80 to enhance fitting. In some embodiments, the surface of spray head 142 may include alternate surface configurations, such as, for example, smooth, rough, arcuate, undulating, mesh, porous, semi-porous, dimpled and/or textured according to the requirements of a particular application.

In other exemplary embodiments of the device, as shown in FIG. 4A, the longitudinally-disposed irrigation channels, such as, for example, channels 143 within spray head 142 may be a combination of the channels that run through the interior of spray head 142, as with channels 142, and channels that run along the circumference of spray head 142, as with channels 143. In one embodiment, the channels which perforate the proximal face of the rigid distal spray head 142, continue some distance within the body of the rigid head, and then “turn” laterally, and become grooves around the circumference of the head unit. In a further embodiment the channels are located proximally on the lateral circumference of the head, and penetrate “inwards” to become channels within the head, which then perforate the distal face of the rigid head unit. In another embodiment, the channels begin at proximal face 145 and twist in a helical fashion along the length of spray head 142 to distal face 144. Any such combination of these various combinations is included within the scope of the present disclosure.

It should be understood that, in some embodiments, the fluid source is not directly connected to the rigid head. Rather, there is a space between the fluid source or tube and the rigid head, such that the rigid head and its channels guide the fluid in and/or out of the working channel and functions as a spray head. In some embodiments, the rigid head is connected to a guidewire for longitudinal movement in and out of the working channel of the endoscope.

Similar to spray heads 42, 142, spray head 242 extends along a longitudinal axis between a distal face 244 and a proximal face 245. Spray head 242 includes a diameter-altering means, such as, for example, flared portion 246 adjacent distal face 244. In some embodiments, flared portion 246 has a diameter larger than the diameter of the rest of spray head 242 so as to facilitate complete sealing of the distal exit of working channel 80. In this way, clearance of working channel 80 may be achieved. Spray head 242 is constructed with lateral grooves 243 which, similar to channels 143, are formed as grooves on the outer surface of spray head 242.

As shown in FIG. 6, channels 143 do not extend along the entire length of spray head 242. In one embodiment, channels 243 comprise a circular cross-section. In some embodiments, channels 243 can be variously configured, such as, for example, oval, oblong, triangular, square, polygonal, irregular, uniform, non-uniform and/or tapered.

In use, following aspiration, spray head 242 may be withdrawn in a proximal direction, such that the flared portion 246 of spray head 242 seals the end of working channel 80 but still remains outside of working channel 80. Since channels 243 in this spray head 242 do not provide a fluid passage all the way to distal face 244, working channel 80 becomes completely isolated from the colonic lumen that lies beyond the end of the working channel. Thus, negative pressure applied to the working channel 80 results in clearance of the liquid and solid contents of working channel 80, and prevent blockage thereof.

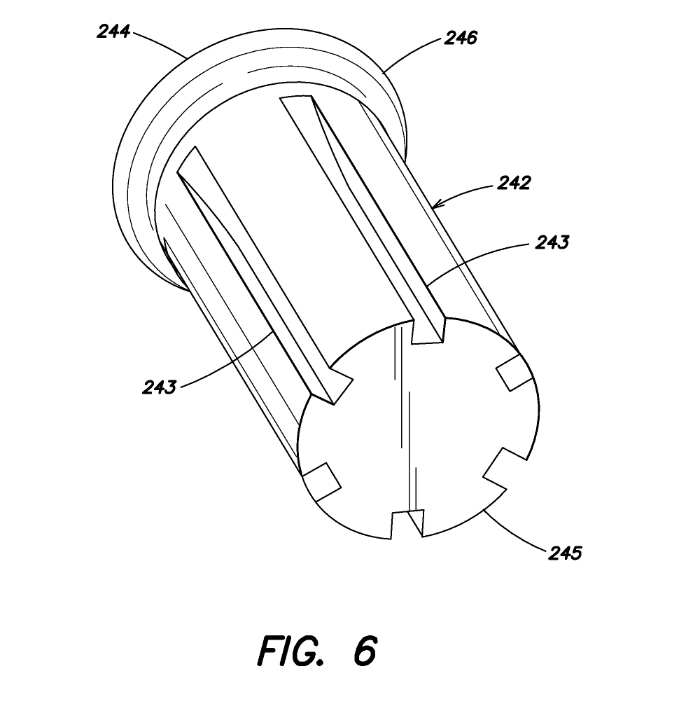

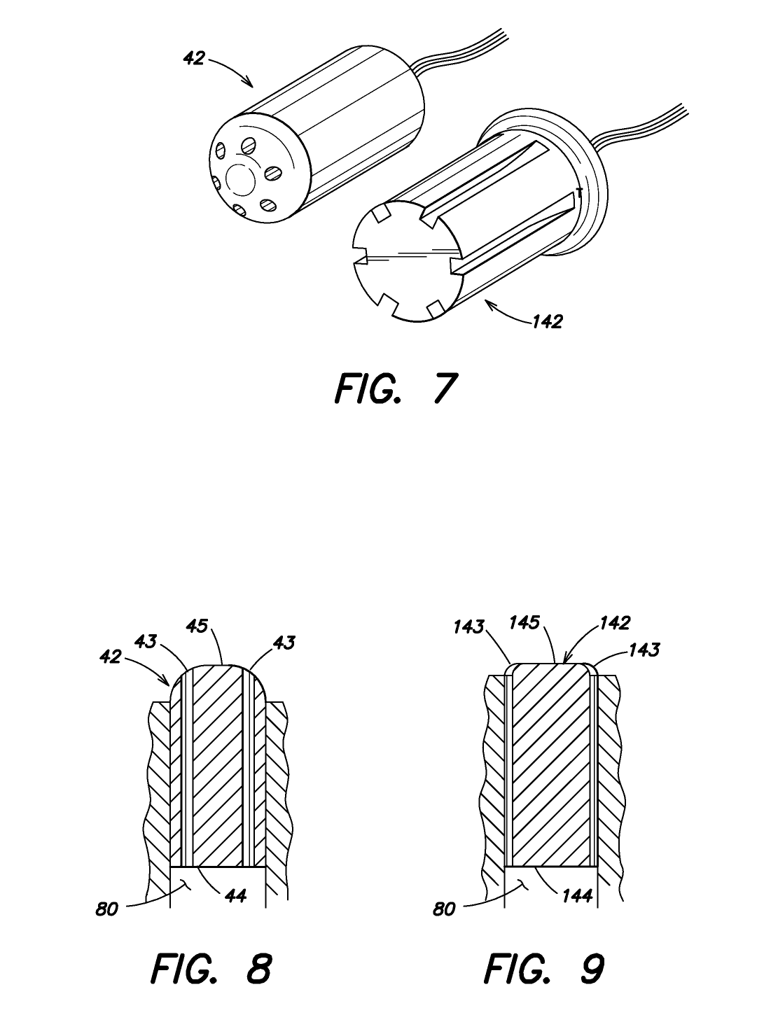

In one embodiment, spray head 242 comprises a generally cylindrical shape and flared portion 246 (this can also be referred to as a rim) that includes a greater diameter than the proximal end of spray head 242. In various embodiments, the diameter of spray head 242 may increase or decrease along its length, as may be required by a particular application. In one embodiment, spray head 242 has a frictional surface configuration for engagement with working channel 80 to enhance fitting. In some embodiments, the surface of spray head 242 may include alternate surface configurations, such as, for example, smooth, rough, arcuate, undulating, mesh, porous, semi-porous, dimpled and/or textured according to the requirements of a particular application. In some embodiments, when the cylindrical shape and flared portion 246 is out of the working channel of the endoscope, fluid will be guided out of channels 243. In some embodiments, when cylindrical shape and flared portion 246 is within the working channel of the endoscope, fluid will be prevented from passing it as the diameter of the cylindrical shape and flared portion 246 will have a diameter just smaller than the diameter of the working channel and fit snuggly within it to create a seal with the inner wall of the working channel. In this way, fluid will flow in the working channel but up to the cylindrical shape and flared portion 246, which allows the working channel of the endoscope to be cleaned. It will be understood, in some embodiments, that the guidewire can be disposed adjacent the cylindrical shape and flared portion 246 (as shown in FIG. 7) so that the spray head 242 can be moved in and out of the endoscope or it can be disposed or attached to proximal face 245 for movement of spray head 242 in and out of the endoscope.

FIGS. 7-9 illustrate in longitudinal section view, the manner in which two spray heads 42, 142 may be used for irrigating the colonic lumen distal to the distal extremity of the working channel. In one embodiment, rigid head 42 is advanced within working channel 80 of a standard, unmodified endoscope, such that distal face 44 of rigid head 42 becomes located a small distance (e.g. a few millimeters) beyond the distal extremity working channel 80. The close fit of the rigid head 42 within working channel 80, and the use of the entire volume of working channel 80 as a fluid conduit, creates pressure of irrigation fluid that results in the expulsion of a fluid spray through the longitudinally-disposed channels 43 into the colonic lumen beyond the distal tip of the endoscope.

In one embodiment, spray head 142 is advanced within working channel 80 of a standard, unmodified endoscope, similarly to rigid head 42. In this embodiment, however, the irrigation fluid is forced through channels 143 formed between the laterally-placed grooves along the circumference of spray head 142 and the inner wall of working channel 80. In the cases of both distal head variants shown in FIGS. 7 and 8, aspiration is achieved by advancing rigid head 42, 142 distally such that its entire length comes to rest within the colonic lumen, beyond the end of working channel 80. In this way, the entire volume of working channel 80 becomes available for the purpose of aspiration of the fluid and solid contents of the colonic cavity that had been subjected to fluid irrigation.

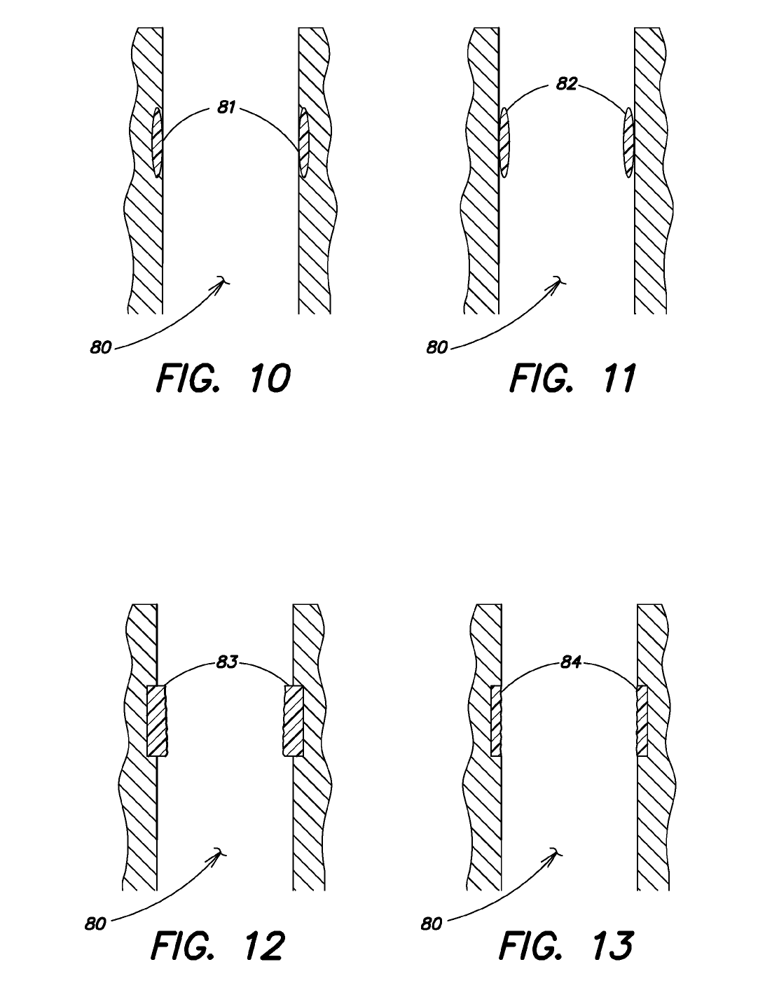

In another embodiment, the entirely rigid head unit 42 (having a length in the range of 2-20 mm) may be used in conjunction with an endoscope in which the inner wall of the distal portion of the working channel 80 has been modified to incorporate diameter-modifying means or elements, as shown in FIGS. 10-15. For example, in one embodiment, as shown in FIG. 10, the elastically deformable element or layer comprises an inflatable balloon 81 or other mechanical means for changing the internal diameter of the working channel. Balloon 81 is configured to move between an inflated configuration and a deflated configuration, wherein the inflated configuration has a larger inner diameter that causes the working channel 80 to have a smaller diameter than when the balloon 81 is deflated or in the deflated configuration, where the working channel 80 will have a larger diameter. Balloon 81 is affixed and/or embedded into the side wall of working channel 80 such that upon inflation of balloon 81, the inner diameter of working channel 80 decreases so as to create a tight fit with rigid head 42. The tolerance created with respect to the diameter d1 of rigid head 42 is sufficiently small to optimally permit pressure build up and irrigation of the colonic lumen and/or the working channel of the endoscope when the rigid head 42 contacts the balloon 81 in the inflated configuration. The tight fit between the rigid head and the balloon will prevent or reduce fluid flow past the rigid head and the balloon and thus the working channel can be cleaned or suctioned. When the balloon is deflated, fluid flow will be increased past the balloon and the working channel and/or patient tissue can be cleaned.

As with the first embodiment described above, the diameter of rigid head 42 in this embodiment should also be such that it permits low friction during its insertion and removal through the working channel and in/out of working channel 80 of the endoscope. In order to enable adequate irrigation, the rigid body should seal at least 60% of the working channel 80 outlet surface of the endoscope. The optimal tolerance is achieved by constructing rigid head 42 such that its outer diameter should be smaller than the working channel diameter by a 0.1 mm or less. In some embodiments, the diameter of rigid head 42 should be less than 0.1 mm smaller than the inner diameter of working channel 80 to accommodate balloon 81.

In various embodiments, working channel 80 of the endoscope is modified such that it comprises means to alter the diameter of said channel in its distal-most portion. These means may be of two functionally-distinct kinds: static means or dynamic means which permit the user to control the endoscope working channel diameter either manually or digitally in order to provide an optimal fit with the rigid head diameter. This may be achieved by an integrated expandable element which can be inflated/deflated within the distal part of the colonoscope working channel; a moveable inner portion to change the inner diameter at a specific location, and/or an integrated lever mechanism that when one side of which is engaged by the rigid head element, the other side of said mechanism applies mechanical pressure to a flexible distal portion of the working channel.

In one embodiment, as shown in FIG. 11, the inner surface of working channel 80 comprises a ring 82 configured to create a tight fit with the outer surface of rigid head 42. Ring 82 has a thickness such that the diameter of the inner surface of ring 82 is smaller than the diameter of the outer thickness of ring 82. In one embodiment, ring 82 has a frictional surface configuration for engagement with rigid head 42 to enhance fitting. In some embodiments, the surface of ring 82 may include alternate surface configurations, such as, for example, smooth, rough, arcuate, undulating, mesh, porous, semi-porous, dimpled and/or textured according to the requirements of a particular application.

In one embodiment, as shown in FIG. 12, the inner surface of working channel 80 comprises a flexible portion 83 configured to create a tight fit with the outer surface of rigid head 42. Flexible portion 83 has elastic properties to conform to the shape of rigid head 42. Flexible portion 83 is embedded into the inner wall of working channel 80 and has a thickness such that the diameter of the inner surface of flexible portion 83 is smaller than the diameter of the working channel 80. In one embodiment, flexible portion 83 has a frictional surface configuration for engagement with rigid head 42 to enhance fitting. In some embodiments, the surface of flexible portion 83 may include alternate surface configurations, such as, for example, smooth, rough, arcuate, undulating, mesh, porous, semi-porous, dimpled and/or textured according to the requirements of a particular application. In some embodiments, the flexible portion can be a spring, clip, projection, ridge or the like that can change configuration according to contact with the rigid head.

In one embodiment, as shown in FIG. 13, the inner surface of working channel 80 comprises a flexible portion 84 configured to create a tight fit with the outer surface of rigid head 42. Flexible portion 84 has elastic properties to conform to the shape of rigid head 42. Flexible portion 84 is embedded into the inner wall of working channel 80 and has an inner surface such that the inner surface of flexible portion 84 is flush with the inner surface of working channel 84. In one embodiment, flexible portion 84 has a frictional surface configuration for engagement with rigid head 42 to enhance fitting. In some embodiments, the surface of flexible portion 84 may include alternate surface configurations, such as, for example, smooth, rough, arcuate, undulating, mesh, porous, semi-porous, dimpled and/or textured according to the requirements of a particular application. It will be understood that the flexible portion 84 can be disposed parallel or substantially parallel to each other as shown in FIG. 13. However, other arrangements are possible, for example, the working channel can have one flexible portion 84, or one or more flexible portion 84 being disposed above or below each other on one part of the working channel or the flexible portion 84 can be disposed at discrete regions of the working channel.

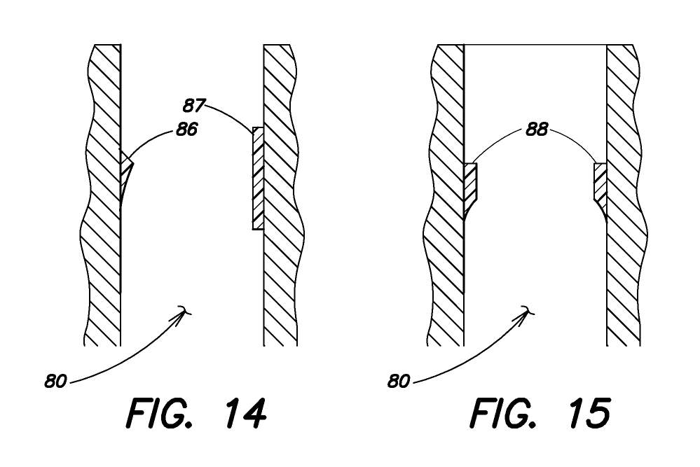

In one embodiment, as shown in FIG. 14, the inner surface of working channel 80 comprises an angled surface 86 and a contact surface 87 configured to create a tight fit with the outer surface of rigid head 42. Angled surface 86 is configured to engage rigid head 42 as rigid head 42 is advanced distally along working channel 80. As rigid head 42 is advanced distally, angled surface 86 pushes rigid head 42 into contact surface 87 such that rigid head 42 is fit tightly between angled surface 86 and contact surface 87. In one embodiment, angled surface 86 and/or contact surface 87 have elastic properties to conform to the shape of rigid head 42. In one embodiment, angled surface 86 and/or contact surface 87 has a frictional surface configuration for engagement with rigid head 42 to enhance fitting. In some embodiments, the surface of angled surface 86 and/or contact surface 87 may include alternate surface configurations, such as, for example, smooth, rough, arcuate, undulating, mesh, porous, semi-porous, dimpled and/or textured according to the requirements of a particular application.

In one embodiment, as shown in FIG. 15, the inner surface of working channel 80 comprises angled protrusions 88 configured to create a tight fit with the outer surface of rigid head 42. Angled protrusions 88 are configured to engage rigid head 42 as rigid head 42 is advanced distally along working channel 80. As rigid head 42 is advanced distally, angled protrusions 88 give resistance to rigid head 42 from opposing sides such that rigid head 42 is fit tightly between angled protrusions 88. In one embodiment, angled protrusions 88 have elastic properties to conform to the shape of rigid head 42. In one embodiment, angled protrusions 88 are depressible into the inner wall of working channel 80 to conform to the diameter of rigid head 42. In one embodiment, angled protrusions 88 have a frictional surface configuration for engagement with rigid head 42 to enhance fitting. In some embodiments, the surface of angled protrusions 88 may include alternate surface configurations, such as, for example, smooth, rough, arcuate, undulating, mesh, porous, semi-porous, dimpled and/or textured according to the requirements of a particular application.

In one embodiment, as shown in FIG. 13 the inner surface of working channel 80 comprises lining configured to create a tight fit with the outer surface of rigid head 42.

Rigid head 42 is capable of being moved between the various above-described conformations by virtue of possessing one or more structural or functional features that permit the dimensions of rigid head 42 to be altered by the operator, for example, by using linear shift of rigid head 42 from inside the endoscope working channel to outside and vice versa. Device 10, is not, however, limited to such a mechanism but rather encompasses further embodiments that may include other mechanical mechanism or inflatable mechanisms that can be used to alter the spray head conformation and the use of flexible resin silicone or rubbers.

Rigid head 42 effectively functions as a perforated plug that may be caused to partially or completely block the distal exit of passageway 32. Thus, when partially blocking said distal exit, irrigation fluid is supplied through passageway 32, the fluid being caused to exit rigid head 42 at a higher pressure, in the form of a jet scatter directed towards the region of the body cavity (e.g., colon) located immediately distal to the distal end of passageway 32.

The irrigation fluid may be supplied to rigid head 42 in the following manner: the irrigation fluids fed into passageway 32 using a positive pressure water pump (peristaltic, centrifugal pump, dosing pump, gearwheel pump, etc.) at a pump outlet pressure of between 2 and 10 atmospheres, resulting in a pressure range of 2-8 atmospheres in the outlet nozzle. The flow rate may range between 0.2 and 2.0 l/min. Sealing elements, adapters and connectors using standard Luer components may be used. It is to be emphasized that the abovementioned pressure and flow parameters are for the purpose of illustration only.

In use, rigid head 42, while in its second, expanded conformation may be moved distally from its seated position over the exit of passageway 32 (as described immediately hereinabove) such that free fluid transfer between passageway 32 and the region of the body cavity located beyond the distal end thereof is once again possible. In this state, passageway 32 may be employed as a suction channel for the aspiration of fluid and solid debris from the body cavity, as well as for the passage of endoscopic tools.

It is to be emphasized that the potential use of passageway 32 as an aspiration channel is facilitated by the fact that rigid head 42 is mounted on the aforementioned very small diameter guidewire 38 made of either metal or plastic resin, rather than on a fluid-supply catheter that would occupy a correspondingly larger fraction of the available working channel volume. Guidewire 38 is sufficiently rigid that it allows the operator to advance rigid head 42 through passageway 32 and out into the body passage lumen. However, it also needs to be sufficiently flexible in order to negotiate bends and convolutions within said body passage.

In some embodiments, rigid head 42 comprises an external O-ring constructed of a flexible material such as silicone. The presence of this O-ring assists in providing a smooth transition between the various head unit positions when moving from one operational mode to another. The use of such an O-ring is of particular value when device 10 is used in conjunction with an endoscope that has an internal taper at the distal end of the working channel. In such a case, the passage of rigid head 42 through the working channel will largely be friction-free until rigid head 42 enters the narrowed distal portion of the working channel.

Device 10 may be used for irrigation and cleansing of the working channel of an endoscopic instrument, in a manner such that blockages of said channel by fecal material may be prevented or removed. Thus, whenever the working channel becomes blocked by feces (and/or other solid and semi-solid material), or alternatively before it is thus blocked, rigid head 42 is mounted on a flexible wire, such as, for example, guidewire 38, which in turn passes through a hollow tube, such as, for example, an inner tube 30, through which irrigation fluid can pass. Tube 30 may have side apertures formed along its entire length or a portion thereof and/or an aperture at the distal end.

In addition to the fluid causing reduction in the size of the solid debris an additional usage of the fluid is to create a positive hydrostatic pressure force (e.g. between 3 and 8 atmospheres) in order to help push the feces and blockage backwards, in addition to the vacuum force which is limited to a maximum of −1 atmosphere of pressure. In practice, the vacuum pressure actually achieved at the distal part of the endoscope may be much less than −1 atm. In this configuration it is necessary to seal the distal end of the endoscope.

In some embodiments, device 10 includes means for assisting an operator to recognize and detect the position of rigid head 42 during use. In one such embodiment, one half of a ratchet mechanism is fitted to the distal face of the endoscope adjoining the distal exit of passageway 32. A complementary ratchet surface is incorporated into the proximal face of rigid head 42 such that when rigid head 42 is brought into contact with the distal exit of passageway 32, a clicking sound (caused by the ratchet mechanism) is emitted, thus informing the operator that the rigid head 42 is in close apposition with the endoscope distal face. Other embodiments incorporate different mechanisms for signaling the position rigid head 42, including remote sensors or transmitters located on the distal face of the endoscope that communicate with receivers or transmitters located on handle 12.

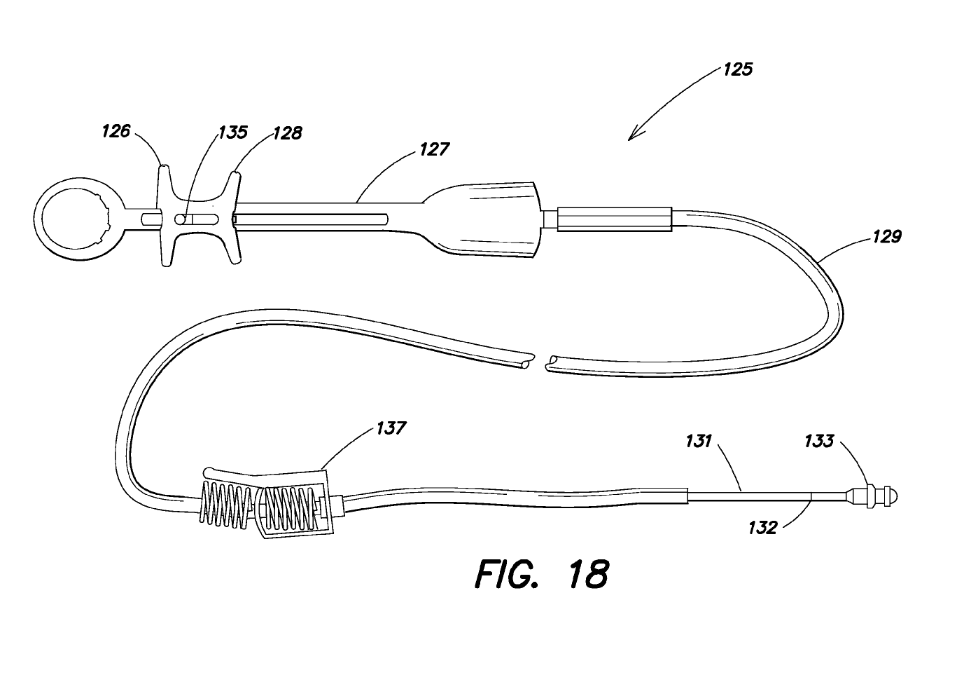

In another embodiment, shown in FIG. 18, there is a body passage device 125 comprising a housing 127 configured to receive a guidewire, the housing 127 has a longitudinal axis, and a first guidewire adjuster is shown contacting the housing and having a proximal end 126 and a distal end 128, the first guidewire adjuster configured to extend the guidewire at a first length shown along the longitudinal axis of the housing; and a second guidewire adjuster 135, which is configured to contact the proximal end and/or the distal end of the first guidewire adjuster and/or guidewire and extend the guidewire at a second length relative to the first guidewire adjuster along the longitudinal axis of housing 127. The first guidewire adjuster is shown as a slider that the user can place their thumb through the thumb ring, which aids in moving slider and guidewire forward or backward. In this way, guide wire and spray head 133 (or plug) can be precisely manipulated in and out of the endoscope working channel or proximal or distal to patient tissue or a body cavity (e.g., colon, esophagus, etc.) so that the target area can be cleaned or irrigated with a fluid (e.g., sterile water, saline, normal saline, dextrose, etc.). Fluid will travel out spray head 133. In some embodiments, spray head 133 has deformable channels (not shown) that guide water out of the working channel of the endoscope. When the channels are open, water is guided to the specific target area. When the channels are closed, for example, the channels collapse on themselves when spray 133 head is inside the working channel of the endoscope, spray head 133 will also function as a deformable plug and prevent the fluid from going beyond spray head 133. In this way, debris or other material inside the working channel of the endoscope can be cleaned or irrigated as well. The guide wire passes through the channel of outer tube 129. In some embodiments, outer tube 129 can be a partial length outer tube and not run continuously with the guidewire as shown in the current embodiment.

To reduce or eliminate this unwanted lateral and/or vertical movement, in some embodiments, body passage device 125 comprises a stabilizing member 132. More particularly, body passage device 125 has an outer tube 129 having proximal end in the direction of the second guidewire adjuster 135 and distal end in the direction of spray head 133 and a longitudinal axis disposed therebetween. Outer tube 129 has a channel therein extending along the longitudinal axis of outer tube 129. A guidewire disposed in the channel, which is configured to extend through the proximal end of the outer tube and through and beyond the distal end of the outer tube and a stabilizing member shown as an inner tube 131 is configured to prevent lateral and/or vertical movement of the guidewire relative to the channel of outer tube 129.

By placing an inner tube 131 coaxial with outer tube 129, which may be a full length or partial length with outer tube 129, or extend more than the length of outer tube 129, inner tube 131 increases the diameter of the device in the working channel and stabilizes the guidewire and spray head 131 to reduce unwanted lateral and/or vertical movement of the guidewire. Inner tube 131 can be disposed within the channel of outer tube 129.

In some embodiments, in addition to inner tube 131 or in place of it, body passage device 125 can have a stabilizing member, such as, for example, a ring, rib or spring, disposed in the channel of inner tube 131, which is configured to contact the guidewire to reduce lateral and/or vertical movement of the guidewire. In some embodiments, the ring, rib, or spring can directly contact the guidewire to reduce or eliminate unwanted lateral and/or vertical movement of the guidewire. In some embodiments, the ring, rib, or spring can contact inner tube 131 and/or outer tube 129 to reduce or eliminate lateral and/or vertical movement of the guidewire.

In some embodiments, body passage device 125 comprises a valve or clamp 137 to shut off water flow entering the inner and/or outer tube. The portions of the device containing the guidewire, inner and/or outer tube can be inserted into a working channel of an endoscope to clean or irrigate the working channel or patient tissue.

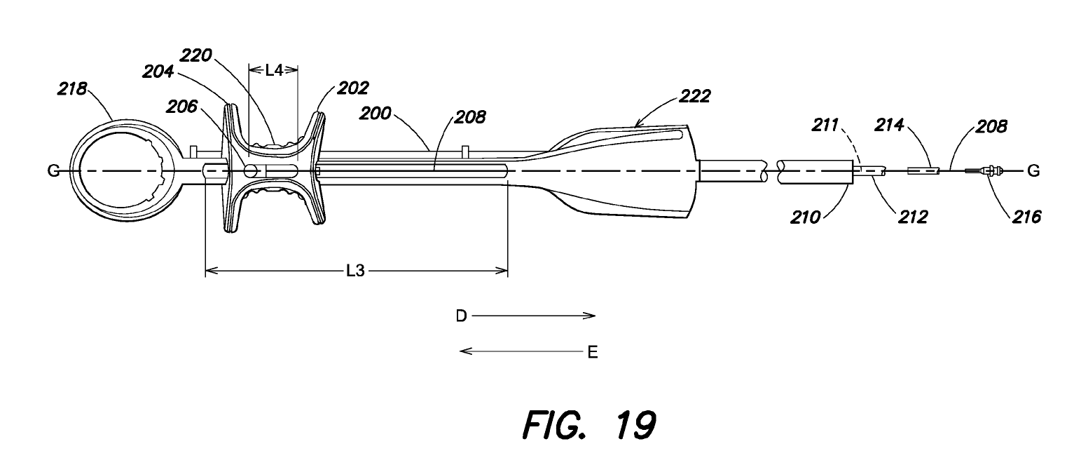

In one embodiment, outer tube 22, stabilizing member 21, guide wire 38 are disposed in working channel 53 of an endoscope 51. In the embodiment shown, the rigid head 42 and a plug are deformable (e.g., they are made from, for example, elastomeric material) and they are shown being retracted into the working channel 53 of the endoscope 51. This can be accomplished by moving a first guidewire adjuster (220 of FIG. 19) and/or second guidewire adjuster (206 of FIG. 19) in the direction of arrow E of FIG. 19. In this way, guide wire 38 and rigid head 42 and the plug can be precisely manipulated in and out of the endoscope working channel so that the target area can be cleaned or irrigated with a fluid (e.g., sterile water, saline, normal saline, dextrose, etc.). Channels 41 of rigid head 42 will deform or collapse as rigid head 42 and the plug are retracted into the working channel due to the fact that the plug, rigid head 42 and/or channels 41 will expand outside of the working channel 53. As the plug, rigid head 42 and/or channels 41 retract into the working channel of the endoscope; channels 41 will contract or collapse or close on themselves due to the snug fit within the working channel and create a seal, which will prevent fluid flow in the working channel. In some embodiments, the plug is also deformable, and will seal the working channel and prevent forward fluid flow 49 in the working channel beyond that of the channels 41 of rigid head 42. This will create back fluid pressure shown by arrow 47 that can irrigate and/or clean debris in the working channel.

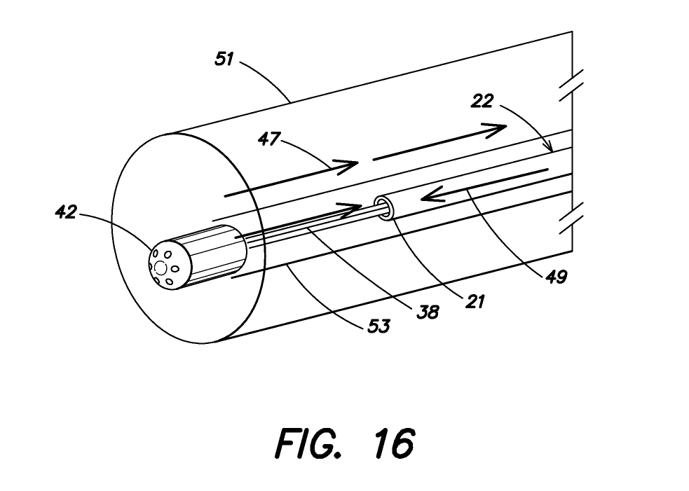

For example, in FIG. 16, the stabilizing member 21 is shown as a ring which compresses on guidewire 38 and reduces or eliminates unwanted lateral and/or vertical movement of guidewire 38.

Turning to FIG. 19 there are illustrated components of a body passage cleaning device 222. Body passage device 222 comprises a housing 200 configured to receive a guidewire 208, the housing 200 having a longitudinal axis G. A first guidewire adjuster 220 is shown contacting housing 200 and having a proximal end 204 and a distal end 202. First guidewire adjuster 220 is configured to extend guidewire 208 a first length, shown as L3 along the longitudinal axis G of housing 200. A second guidewire adjuster 206 is configured to contact proximal end 204 and/or distal end 202 first guidewire adjuster 220 and/or guidewire 208. Guidewire 208 extends a second length L4 relative to first guidewire adjuster 220 along the longitudinal axis of housing 200. First guidewire adjuster 220 is shown as a slider that the user can place their thumb into thumb ring 218, which aids in moving the slider and guidewire 208 forward (shown in the direction of arrow D) and backward (shown in the direction arrow E). In this way, guide wire 208 and a spray head 216 (or plug) can be precisely manipulated in and out of the endoscope working channel or proximal or distal to patient tissue or a body cavity (e.g., colon, esophagus, etc.) such that the target area can be cleaned, aspirated, or irrigated with a fluid (e.g., sterile water, saline, normal saline, dextrose, etc.). Fluid will travel out of spray head 216. In some embodiments, spray head 216 can have deformable channels (not shown) that guide water out of the working channel of the endoscope. When the channels are open, water is guided to the specific target area. When the channels are closed, from for example, the collapse of the channels on themselves when the spray head is inside the working channel of the endoscope, spray head 216 will also function as a deformable plug and prevent the fluid from going beyond spray head 216. In this way, debris or other material inside the working channel of the endoscope can be cleaned or irrigated as well. The guide wire passes through the channel of an outer tube 210. In some embodiments, outer tube 210 can be a partial length outer tube and not run continuously with guidewire 208 as shown in the current embodiment.

If additional length is needed for guidewire 208 and/or spray head 216, due to, for example, the various lengths of endoscope working channels, including those that are the same makes and models, or if additional length is needed to irrigate or clean patient tissue, the user moves or slides second guidewire adjuster 206 in the direction shown by arrow D or forward to cause guidewire 208 and spray head 216 to move forward. Therefore, precise irrigation or cleaning can be achieved if needed, when water passes out of outer tube 210 and the stream is guided by spray head 216 to the target area. It will be understood that first guidewire adjuster 220 or second guidewire adjuster 206 can be moved forward as shown in arrow D or backward shown in arrow E to control the guidewire 208 and/or spray head 216. In this way, precise control of the cleaning and/or irrigation can be achieved.

In one embodiment, second guidewire adjuster 206 is disposed within first guidewire adjuster 220 and first guidewire adjuster 220 is slidably mounted along the longitudinal axis of housing 200 to extend guidewire 208 the first length. The end of guidewire 208 can directly contact first guidewire adjuster 220. In the embodiment shown, guidewire 208 can be anchored at one end of second guidewire adjuster 206 and first guidewire adjuster 220 directly contacts second guidewire adjuster 206, which directly contacts guidewire 208. Movement (e.g., sliding) of first guidewire adjuster 220 along the longitudinal axis of housing 220 in the forward direction D causes second guidewire adjuster 206 to move in direction D. If additional length L4 is needed for guidewire 208 due to, for example, using an endoscope with a different length working channel, a user will move or slide first guidewire adjuster 220 forward to length L3, then second guidewire adjuster 206 can further be moved or slid to the additional length L4. The guidewire adjusters can be universally configured for both right and left hand users. First guidewire adjuster 220 can also be smaller in length and easier to manipulate based on the user's hand and finger size, as second guidewire adjuster 206 is available, if additional length is needed. In this way, the guidewire adjusters can fit the user's hands comfortably.

In one embodiment, first guidewire adjuster 220 and second guidewire adjuster 206 are slidably mounted with housing 200. However, other mountings are possible. In one embodiment, first guidewire adjuster 220 can engage guidewire 208 until it slides or moves all or a part of first length L3, then second guidewire adjuster 206 can engage guidewire 208 to move or slide it all or part of the additional length or the second length L4. In this way, guidewire 208 and/or spray head 216 can be moved into discrete positions and the user can comfortably slide or move guidewire 208 to those positions.

In one embodiment, second guidewire adjuster 206 is disposed within first guidewire adjuster 220 and parallel or substantially parallel to it so that moving one will move the other. However, it will be understood by those skilled in the art that other configurations are possible and the guidewire adjuster 220 can be orthogonal or perpendicular to second guidewire adjuster 206. In the embodiment, guidewire 208 is anchored in second guidewire adjuster 206 and first guidewire adjuster's 220 proximal end 206, does not directly contact guidewire 208. In other embodiments, both first guidewire adjuster 220 and second guidewire adjuster 206 can both contact guidewire 208.

Body passage device 222, because portions of it have a smaller diameter than the diameter of the working channel of the endoscope, often at times guidewire 208 and spray head 216 have unwanted lateral and vertical motion, which may lead to imprecise cleaning and/or irrigation.

To reduce or eliminate this unwanted lateral and/or vertical movement, in some embodiments, body passage device 222 comprises a stabilizing member 214. More particularly, body passage device 222 has an outer tube 210 having a proximal end in the direction of first guidewire adjuster 220 and a distal end in the direction of spray head 216, and a longitudinal axis disposed therebetween. Outer tube 210 has a channel 211 therein extending along the longitudinal axis of outer tube 210. Guidewire 208 is disposed in channel 211, which is configured to extend through the proximal end of outer tube 210 and through and beyond the distal end of outer tube 210. Stabilizing member 214 is configured to prevent lateral and/or vertical movement of guidewire 208 relative to channel 211. By placing inner tube 212 coaxially with outer tube 210, which may be a full length or partial length with the outer tube 210, or extend more than the length of the outer tube 210, inner tube 212 increases the diameter of the device in the working channel and stabilizes guidewire 208 and spray head 216 to reduce unwanted lateral and/or vertical movement of guidewire 208. It will be understood that inner tube 212 can be disposed within outer tube channel 211.

In some embodiments, inner tube 212 is the same length as outer tube 210. In some embodiments, inner tube 212 is a smaller length than outer tube 210. In some embodiments, inner tube 212 is larger than the length of outer tube 210. In some embodiments, inner tube 212 extends further along longitudinal axis G than outer tube 210.

In some embodiments, in addition to inner tube 212 or in place of it, body passage device 222 can have a different stabilizing member that comprises a ring, rib, or spring disposed in channel 211 of outer tube 210, which is configured to contact guidewire 208 to reduce lateral and/or vertical movement of guidewire 208. In some embodiments, the ring, rib, or spring; can directly contact guidewire 208 to reduce or eliminate unwanted lateral and/or vertical movement of guidewire 208. In some embodiments, the ring, rib, or spring can contact inner tube 212 and/or outer tube 210 to reduce or eliminate lateral and/or vertical movement of guidewire 208. For example, stabilizing member 214 can be disposed within channel 211 of outer tube 210, and contact outer tube 210 and guidewire 208, to reduce or eliminate lateral and/or vertical movement of guidewire 208.

It will be understood by those of ordinary skill in the art that the body passage device and one or more of its components can be sterilized and reused. Alternatively, and more preferred, one or more components of the body passage device can be disposable and disposed of after single use.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A rigid head for use in a working channel of an endoscope, the rigid head comprising: a body having a proximal end, a distal end and a longitudinal axis, the body having at least one channel disposed along the longitudinal axis configured to guide fluid in or out of the working channel, wherein the rigid head is configured for disposal within a working channel of an endoscope, the at least one channel configured to guide fluid in and out of the working channel of the endoscope.

2. A rigid head according to claim 1, wherein the head is connectable to a guidewire.

3. A rigid head according to claim 1, wherein the at least one channel extends through the interior of the body.

4. A rigid head according to claim 1, wherein the at least one channel comprises elongate slots spaced along the periphery of the body.

5. A rigid head according to claim 1, wherein the at least one channel extends through the interior of the body and comprises elongate slots spaced along the periphery of the body.

6. A rigid head according to claim 1, wherein (i) the at least one channel is spaced evenly in a circular pattern about the distal end; (ii) the at least one channel twists in a helical configuration from the proximal end to the distal end; or (iii) the distal end comprises a bullet shape configured for insertion in and out of the working channel of the endoscope.

7. A rigid head according to claim 1, wherein the body comprises an outer diameter that is smaller than a diameter of the working channel.

8. A rigid head according to claim 1, wherein the body comprises an outer diameter between about 1.9 mm to about 5.9 mm.

9. A rigid head according to claim 1, wherein the proximal surface is configured to seal at least 60% of an outlet surface of the working channel.

10. A rigid head according to claim 1, wherein the working channel is configured to discharge fluid to a surgical site.

11. A rigid head according to claim 1, further comprising an expanded head region adjacent the proximal end configured to create a tight fit between the rigid head and an inner surface of the working channel to create a back pressure within the working channel.

12. A system for cleansing tissue, comprising:
a rigid head unit comprising a body having a proximal face, a distal face and a channel extending between the proximal face and the distal face, the head defining a longitudinal axis between the proximal face and the distal face, the head being attached to a distal end of a guidewire extending from an irrigation tool; and
a working channel extending between the rigid head and the irrigation tool,
wherein the proximal face is connectable to the working channel of the surgical tool for transmission of matter.

13. A system for cleansing tissue according to claim 12, wherein the channel extends through the interior of the body.

14. A system for cleansing tissue according to claim 12, wherein the channel comprises elongate rounded slots spaced along the periphery of the body.

15. A system for cleansing tissue according to claim 12, wherein the head unit comprises a substantially cylindrical cross-section along its length, the body having an outer diameter smaller than a diameter of an inner surface of the working channel.

16. A system for cleansing tissue according to claim 12, wherein the head unit comprises a substantially cylindrical cross-section along its length, the body having an outer diameter between about 1.9 mm to about 5.9 mm.

17. A system for cleansing tissue according to claim 12, wherein the working channel comprises an element on an inner surface to provide a tight fit between an outer surface of the rigid head and the working channel.

18. A system for cleansing tissue according to claim 17, wherein the element comprises an expandable member configured to be inflated between a first configuration and a second configuration, the first configuration defining a greater inner diameter than an inner diameter of the second configuration.

19. A system for cleansing tissue according to claim 17, wherein the element comprises a ring embedded into the inner surface.

20. A system for cleansing tissue according to claim 17, wherein the element comprises a flexible portion having elastic properties.

21. A system for cleansing tissue according to claim 17, wherein the element comprises an angled surface disposed opposite a contacting surface of the working channel, such that as the rigid head contacts the angled surface, the rigid head is pressed against the contacting surface to create a tight fit between the rigid head and the working channel.

22. A system for cleansing tissue according to claim 17, wherein the element is movable along the inner wall to change an inner diameter of the wall at a specific location.

23. A system for cleansing tissue according to claim 17, wherein the element comprises a gripping surface to maintain a tight fit with the rigid head.

24. A system for cleansing tissue according to claim 12, wherein the irrigation tool includes a housing configured to receive the guidewire, the housing having a longitudinal axis; a first guidewire adjuster contacting the housing and having a proximal end and a distal end, the first guidewire adjuster configured to extend the guidewire a first length along the longitudinal axis of the housing; and a second guidewire adjuster configured to contact the proximal end and/or the distal end of the first guidewire adjuster and/or guidewire and to extend the guidewire a second length along the longitudinal axis of the housing.

25. A kit for cleansing tissue, comprising
an irrigation tool configured to receive a guidewire, the tool comprising a housing having a longitudinal axis; a first guidewire adjuster contacting the housing and having a proximal end and a distal end, the first guidewire adjuster configured to extend the guidewire a first length along the longitudinal axis of the housing; and a second guidewire adjuster configured to contact the proximal end and/or the distal end of the first guidewire adjuster and/or guidewire and to extend the guidewire a second length along the longitudinal axis of the housing; and
a rigid head unit comprising a body having a proximal end and a distal end and a longitudinal axis, the body having at least one channel disposed along the longitudinal axis, the rigid head configured to be disposed within a working channel of an endoscope, the at least one channel configured to guide fluid in or out of the working channel, and the proximal end connectable to the irrigation tool.

26. A method for cleansing tissue or cleansing a working channel of an endoscope, the method comprising inserting the rigid head of claim 1 into a working channel of an endoscope to cleanse a tissue or the working channel of the endo scope.

27. A method of performing an endoscopy of claim 26, wherein the tissue is a colon or esophagus.

28. A kit according to claim 25, further comprising an endoscope, wherein the working channel extends between the rigid head unit and the irrigation tool, the working channel comprising an element on an inner surface to provide a tight fit between an outer surface of the rigid head unit and the element.
